(19) United States
(12) Patent Application Publication
(10) Pub. No.: US 2005/0164173 A1
(43) Pub. Date: Jul. 28, 2005

(54) Combined treatments and methods for treatment of mycoplasma and mycoplasma-like organism infections

(76) Inventor: Luc Montagnier, New York, NY (US)

Correspondence Address: MILDE & HOFFBERG, LLP, 10 BANK STREET, SUITE 460, WHITE PLAINS, NY, 10606, US

(21) Appl. No.: 11/042,567

(22) Filed: Jan. 24, 2005

Related U.S. Application Data
(63) Continuation of application No. 10/141,036, filed on May 7, 2002.
(60) Provisional application No. 60/289,348, filed on May 8, 2001.

(51) Int. Cl. C12Q 1/70; C12Q 1/68; G01N 33/554; G01N 33/569; A61K 31/43; A61K 31/545

(52) U.S. Cl. 435/5; 435/6; 435/7.32; 514/192; 514/200

(57) ABSTRACT

Methods of treating, and pharmacological formulations and administration methods for treating, mycoplasma-like organism infections in pathological situations. Combinations of antibiotics, targeting different mycoplasma metabolic pathways, are combined with antioxidants to reduce oxidative stresses. This treatment improves host cell resistance to oxidative stress while reducing mycoplasma infection levels.

### FIELD OF THE INVENTION

The present invention relates to the field of treatments and methods for treatment of pathology relating to mycoplasma and mycoplasma-like organism infections.

### BACKGROUND OF THE INVENTION

AIDS researches have continually struggled with the question of whether HIV alone causes AIDS, or whether there are other significant etiological factors involved.

For example, some studies have indicated that mycoplasma is a commonly found co-infecting factor with HIV, and have suggested that mycoplasmas may play a significant role in AIDS.

An antibody raised against adhesin from M. genitalium used to bind or attach itself to its target cells, a conserved surface protein among many species of mycoplasma, was found to block infectivity of HIV in some instances, although with variable results. There is sequence homology between adhesin and the gp120 coat protein of HIV. It has been suggested that the antibody against adhesin protein interfered with the mycoplasma's ability to attach itself to target cells, and mycoplasma may play a role in the early part of the viral life cycle. Montagnier L, Berneman D, Guetard D et al. “Inhibition de l'infectiosite de souches prototypes du VIH par des anticorps diriges contre une sequence peptidique de mycoplasme.” Comptes Rendus de L'Academie des Sciences 1990; 311(III): 425-430; Balter M. “Montagnier pursues the Mycoplasma-AIDS link.” Research News. Science 1991: 251-271.

No clinical study published to date has, however, shown antibiotic treatment of infections with identified mycoplasmas provides persistent benefits for HIV or other confirmed viral infection.

Most Mycoplasma species are facultatively anaerobic, and all known species are chemoorganotrophic. The fermentative species of Mycoplasma utilize sugars such as glucose, while non-fermentative species can hydrolyze arginine. Only a few species of mycoplasma can utilize both glucose and arginine to grow. Known mycoplasmas may be grown on complex media, such as Hayflick medium, while fastidious mycoplasmas may be grown on diphasic SP-4 medium.

Many species of Mycoplasma produce weak or clear hemolysis which appears to be due to the secretion of H₂O₂. This H₂O₂ secretion is believed to be responsible for some aspects of the mycoplasmas' pathogenicity. Known mycoplasmas are commonly sensitive to chloramphenicol, tetracyclines and fluoroquinolones.

The known mycoplasmas currently include more than 60 known species, which are differentiated on the basis of various tests, including utilization of glucose and mannose, arginine hydrolysis, phosphatase production, the “film and spots” reaction and haemadsorption.

Mycoplasmas are the smallest and simplest free-living organisms known. Mycoplasmas are not obligatory intracellular microorganisms and are usually found extracellularly, but can be found intracellularly in the infected tissues (Mycoplasma, Eds. Wolfgang, J. J., Willette, H. P., Amos, D. B., Wilfert, C. M., Zinsser Microbiology 19th Ed. 1988, Appleton and Lange, 617-623). Ultrastructurally, mycoplasmas have protoplasm-like internal structures and are bounded by only an outer limited membrane (unit membrane) without a cell wall.

Mycoplasmas cause a variety of diseases in animals, plants and insects. However, the roles of known human mycoplasmas except for M. pneumoniae in causing a typical pneumonia are difficult to assess. On the other hand, mycoplasmas are known to be extraordinary pathogens, capable of causing chronic debilitating diseases and producing a variety of clinical manifestations in animals and frequently suppressing host immune defense mechanisms. The Mycoplasmas, Vol. IV, Razin, S., Barile, M. F., eds., Academic Press, pp. 203-286 (1983).

Recent studies suggest mycoplasmas may play an important role in the progression of AIDS. Lo, S-C., et al., Am. J. Trop. Med. Hyg. 40, 213 (1989); Lo, S-C., et al., Am J. Trop. Med. Hyg. 41, 601 (1989), Montagnier, L., et al., C.R. Acad. Sci. Paris 311, 425 (1990); Chowdhury, M. I. H., et al., Lancet 336, 247 (1990); Bauer, F. A., et al., Hum. Pathol. 22, 63 (1991); “Mycoplasma and AIDS—what connection?” [editorial], Lancet 337, 20 (1991); Lo, S-C., et al., Science 251, 1074 (1991). Reported AIDS-associated mycoplasmas include M. fermentans, M. pirum, and M. penetrans, see U.S. Pat. No. 5,215,914 (Lo, et al. Jun. 1, 1993).

In a prior patent, the inventor hereof suggested that treatment of previously identified mycoplasmas with antibiotics was a possible therapy for HIV infected patients. See, U.S. Pat. No. 5,677,123, expressly incorporated herein by reference. A nucleotide probe was suggested to assist in original diagnosis and determination of treatment endpoint. Nucleotide probes can be made which are species specific, or having a broad reactivity with various mycoplasmas.

### SUMMARY OF THE INVENTION

The present invention provides a method for treating chronic animal or human pathologies associated with infection by a prokaryotic microorganism lacking a rigid cell wall, especially mycoplasmas and mycoplasma-like organisms, by administering an effective amount of an antibiotic pharmacological agent. Advantageously, an effective amount of an antioxidant pharmacological agent is coadministered. While antibiotics alone and antioxidants alone can treat mycoplasma infection, together they act at different targets of the mycoplasma physiology and host pathology and therefore can be advantageously combined. It is noted that, at high doses, antioxidants have been found by the inventor hereof to induce mycoplasma growth, and thus the antioxidant therapy should be optimized to an effective level. The antioxidant pharmacological agent is therefore preferably present in a sufficient amount to reduce oxidative stress on the host organism cells and which is below a level that might be predicted to induce mycoplasma activity. Preferably, the antioxidant is also present in an effective amount to enhance host immune response.

According to the present invention, it is recognized that there may exist organisms related to the classic and previously identified mycoplasmas, but which have not been formally included within the family. As used in the present application, it is understood that these organisms are encompassed by the term mycoplasma, unless particularly distinguished. In general, these mycoplasma like organisms (MLOs) (also referred to herein as mycoplasma related organisms) are difficult to detect and clear, which may result from specific growth conditions and different antibiotic sensitivities. Therefore, it is understood that prior efforts to identify and treat the previously identified mycoplasmas may have been confounded by these MLOs, and reported results thereon may be confused or explained by the presence of these related, though biologically distinct strains.

Another aspect of the invention provides a method of treating human mycoplasma related organism pathology, comprising the steps of administering a drug targeting a specific pharmacological process within the mycoplasma related organism; and maintaining a redox state within a range which reduces mycoplasma related organism metabolism.

The present invention further provides a method for treating an immunocompromised HIV infected individual, comprising the steps of treating the patient with an highly active antiretroviral therapy to partially restore immune function, and interrupting the highly active antiretroviral therapy, and subsequently instituting a maintenance therapy comprising one or more of a therapy selected from the group consisting of antioxidants in a sufficient amount to reduce oxidative stress, and prokaryotic active antibiotics.

The present invention also provides a method for treating an immunocompromised HIV infected individual, comprising the steps of treating the patient with combination highly active antiretroviral and antimycoplasma and/or mycoplasma-like organism therapy to reduce viral load and mycoplasma levels, and interrupting the highly active antiretroviral therapy, and instituting a maintenance therapy comprising a therapy selected from one or more of the group consisting of antioxidants in a sufficient amount to reduce oxidative stress, and prokaryotic active antibiotics, the prokaryotic active antibiotics being selected to have antimycoplasma and/or antimycoplasma-like organism activity.

The present invention further provides a method for treating an immunocompromised patient, comprising the steps of treating the patient with an antibiotic effective against antimycoplasma and/or mycoplasma-like organisms, and administering antioxidants in a sufficient amount to reduce oxidative stress. This immunocompromised state may be due to, for example, neoplastic disease, immunosuppressive therapies, age, viral or bacterial disease, or other types, including idiopathic. The administration of antioxidants in conjunction with antimycoplasma agents is generally preferred, since antioxidants are available having good therapeutic profiles, may enhance patient health through a variety of mechanisms, and are believed to provide increased effect over antimycoplasma antibiotics alone. However, it is also an object of the present invention to provide an antimycoplasma antibiotic therapy for treatment of diseases in which direct mycoplasma involvement in pathology (and a beneficial effect in reducing mycoplasma activity) was previously unrecognized, without requiring combination therapy therefore.

Another aspect of the invention provides a method for treating human mycoplasma related organism pathology, comprising the steps of administering a drug targeting a specific pharmacological process within the mycoplasma related organism; and maintaining an antioxidant level within a range which reduces mycoplasma related organism metabolism.

A free radical scavenger may be useful for deactivating mycoplasma or mycoplasma-like organism transcription activation factors, especially in infections associated with oxidized host intracellular conditions.

The antibiotics active against human mycoplasma include the tetracycline family, for example, minocycline or doxycycline, and the fluoroquinolone family, which includes for example ciprofloxacin and sparfloxacin.

The antioxidants or redox altering agents preferred according to the present invention include reduced glutathione (for example Ultrathione™ from Thyogen Corp., Elmsford, N.Y.), and superoxide dismutase, for example from animal or vegetable sources. One way to determine the optimal antioxidant levels and/or drug combinations is to empirically test cultured mycoplasma with medium having various concentrations of the agents. The mycoplasma growth may be determined, for example, by the size of a uracil-labeled peak in an equilibrium sucrose density gradient.

The present invention also provides a pharmacological dosage form, for administration of a treatment to humans or animals comprising an antibiotic and at least one antioxidant, the at least one antioxidant being present in an efficacious amount to additionally reduce mycoplasma-like organism activity with the antibiotic. A second microbial antibiotic may also be added, wherein the antibiotic and second microbial antibiotic specifically target different metabolic pathways of the mycoplasma-like organism. For example, a combination of doxycycline and ciprofloxacin may be employed with glutathione and ascorbic acid as antioxidants.

The present invention also provides a method for optimizing a pharmacological regimen for treating a human or animal subject infected with an atypical mycoplasma like organism, by inhibiting bacteria in a medium containing an antibiotic active on a cell wall target, and detecting the inhibition of mycoplasma growth by a proposed pharmacological treatment in vitro, as evidenced by the inhibition of uracil-incorporation into supernatant fractions having a density characteristic of mycoplasma. This method may be embodied in a diagnostic kit for use in testing in vitro or in vivo samples. Such a kit consists of an incubation medium comprising the anti-cell wall active antibiotic, as well as labeled uracil or another specific mycoplasma assay reagent, e.g., ELISA, luminescent or fluorescent probes.

The present invention further provides a method of optimizing a pharmacological regimen, comprising detecting a mycoplasma or mycoplasma-like organism infecting host cells, determining a combination of antibiotic and antioxidant effective against the detected organism, and administering the antibiotic and antioxidant in an effective amount, based on the determination, to suppress the organism. Likewise, the present invention also comprises a pharmaceutical formulation in unit dosage form, including the combination of anti-mycoplasma antibiotic and antioxidant, each present in an effective amount, optionally along with other inert or active ingredients. For example, if the patient is or is anticipated to be infected with another organism (e.g., HIV), then the unit dosage form may also comprise an agent in an effective amount targeting that other organism.

It is a still further object of the invention to provide a method for detecting mycoplasma-like organisms in a medium, comprising the steps of immunizing an animal with a purified mycoplasma-like organism preparation, purifying antibodies produced by the animal, and performing an antibody-antigen binding assay of the medium to determine mycoplasma-like organism proteins. Monoclonal antibodies may also be generated against these organisms. The antibodies may thus be monoclonal or polyclonal, and can be obtained from mammals or birds. Advantageously, the immunization may be conducted with polynucleotides as adjuvants.

As an alternative to the uracil incorporation technique for detecting mycoplasma, it is also possible to detect mycoplasma based on their particular metabolic patterns, enzymes and transport proteins. Thus, a specific enzyme may be assayed by the disappearance of substrate or the appearance of a product. These, in turn, may be measured by ELISA, fluorimetric or colorimetric assays. In addition, mycoplasma-associated metabolism, or incorporation of abnormal component nucleic acid bases may also be determined. In addition, nucleic acid precursor transport systems, e.g., uracil, can also be determined.

The present invention also provides a method of detecting a nucleic acid specific for a mycoplasma-like organism, comprising the steps of extracting nucleic acid from a sample, amplifying the nucleic acid from the sample with mycoplasma-like organism specific primers, and analyzing the amplified nucleic acid to detect the mycoplasma-like organism specific nucleic acid. It is noted that the primers selected for mycoplasma-like organisms may differ from those selected for mycoplasmas. This therefore allows a sensitive determination of mycoplasma-like organisms without interference from mycoplasma, which are generally easier to culture in acellular medium.

The present invention also provides a method for detecting a nucleic acid specific for a mycoplasma-like organism associated pathology, comprising the steps of extracting nucleic acid from a sample, amplifying the nucleic acid from the sample with a type-specific mycoplasma-like organism specific primer, and analyzing the amplified nucleic acid to detect the mycoplasma-like organism specific nucleic acid.

While a direct assay for mycoplasma or mycoplasma-like organism nucleic acids is preferred, other methods may be used to identify the presence and type of organism. For example so-called subtractive assays, specific antibody assays, or bioassay.

The mycoplasma or mycoplasma-like organisms may be found circulating in the blood, associated with specific elements, like leukocytes, erythrocytes, platelets or in the plasma. Therefore, if the particular organism is as-yet unidentified, it is important to evaluate each compartment for the organism. In general, the mycoplasma-like organisms are more difficult to culture in cell free medium than the known mycoplasma organisms (for which there are specific culture media, such as SP-4 medium (Tully et al., Science 1977 195, page 892-894)), and therefore special care and attention must be employed in their isolation. Short-term culture in cell free medium is sometimes possible.

t is also possible to produce mycoplasma or mycoplasma-like organisms by inoculating initially healthy lymphocytes with mycoplasma-containing supernatant from putatively infected cells, or directly culturing putatively infected cells, for example in SP-4 medium containing labeled uracil. The supernatant is then subjected to sucrose density centrifugation and a fraction around the 1.21 density fraction, or some lighter fraction, examined. Preferably, the lymphocytes are cultured in both the activated and inactivated states.

Since the DNA sequence corresponding to the ribosomal RNA characteristic of mycoplasmas is highly conserved, a probe directed toward this sequence will have broad sensitivity toward much of the entire class of mycoplasmas. On the other hand, there are highly variable genes which may be used to specifically identify species. If it is desired to detect members of a subclass of mycoplasma-like organisms, specific probes may be mixed, or a probe selective for the subclass selected and employed. If it is desired to determine which species is involved, for example where prognosis or treatment differs, then specific probes may be employed.

In order to make the DNA accessible, the culture of mycoplasmas is advantageously subjected to centrifugation and the centrifugation pellet recovered is digested with a mixture of proteinase K and SDS. Thus, in order to detect the DNA of mycoplasmas, an advantageous procedure according to the invention is carried out as follows: the culture containing the mycoplasmas is centrifuged under conditions leading to the sedimentation of the mycoplasmas and, if necessary, after an amplification step in a culture medium specific for mycoplasmas, the centrifugation pellet is treated in order to recover the DNA, for example with a mixture of proteinase K and SDS, the DNA thus obtained is placed in contact with the reagent comprising at least one mycoplasma or mycoplasma-like organism selective probe.

The present invention allows also the application of these procedures for the detection of any infection due to mycoplasmas, in particular in the blood of a patient. Also included within the framework of the invention is a test for the in vitro study of the sensitivity to one or more given antibiotics of mycoplasmas isolated previously from a biological sample taken from a patient likely to be infected with a AIDS, in conjunction with antioxidant therapies, characterized in that, the cell culture contaminated with the isolated mycoplasma(s) is incubated with various doses of selected antibiotics and antioxidants, this culture is labeled, for example, with radioactive uracil, the radioactivity of each fraction is determined after sedimentation of the supernatant under conditions leading to the separation of the mycoplasmas from the other constituents.

The area of the peaks corresponding to the density of mycoplasmas are then analyzed to determine which are the best combination of drugs, and respective concentrations, to inhibit mycoplasma activity. This will be of value in the treatment of patients infected with the mycoplasma.

The preferred anti-mycoplasma-like organism pharmaceutical formulation comprises, in physical association a combination of glutathione, ascorbic acid, a tetracycline-derivative, a fluoroquinolone, and an orally bioavailable form of superoxide dismutase. Optionally, various known antiviral and/or antiretroviral agents may be added.

### BRIEF DESCRIPTION OF THE DRAWINGS

A preferred embodiment of the invention will now be described with reference to the drawings, in which:

FIG. 1A shows a control curve, wherein the central peak corresponds to the mycoplasma uracil incorporation;

FIG. 1B shows the effect of 50 μg/ml superoxide dismutase;

FIG. 1C shows the effect of 100 μg/ml superoxide dismutase;

FIG. 2A shows a control curve, wherein the central peak corresponds to the mycoplasma uracil incorporation;

FIG. 2B shows the effect of 1 mg/ml glutathione; and

FIG. 2C shows the effect of 3 mg/ml glutathione.

### DETAILED DESCRIPTION OF THE PREFERRED EMBODIMENTS

CEM cell line, derived from lymphoblastic leukemia of a child, were first cultivated for one month in RPMI-1640 plus 10% fetal calf serum (mycoplasma free), in the presence of fluoroquinolone (2 μg/ml), in order to clear any known mycoplasma infection. This treatment is generally considered sufficient to clear out classical mycoplasma, but did not clear mycoplasma-like organisms.

The cells were then cultivated in antibiotic free medium for two weeks.

For the experiments reported below, the cells were kept for five days in fresh medium with an addition of the same volume of fresh medium on the third day. At day four, they received 5 μCi/ml of tritiated uracil for 18 hours. Cells were spun down by low speed centrifugation, and then the supernatant was clarified to centrifugation at 3000 rpm for ten minutes. 0.5 ml of supernatant was layered on a stepwise sucrose density gradient with a range of density of 1.25 to 1.05 gm/ml in a polyethylene tube for a Beckman SW 56 rotor.

The tube was centrifuged for two hours at 35,000 rpm, at 4° C., then the tube was pierced at the bottom and the around 22-30 fractions were collected in polypropylene tubes. Aliquot from some fractions were taken to measure density, using an Abbe refractometer. Aliquots of all fractions were precipitated with TCA in order to measure incorporation of tritiated uracil into soluble material. Radioactivity of each precipitate was measured in a scintillation counter.

### EXAMPLE 1

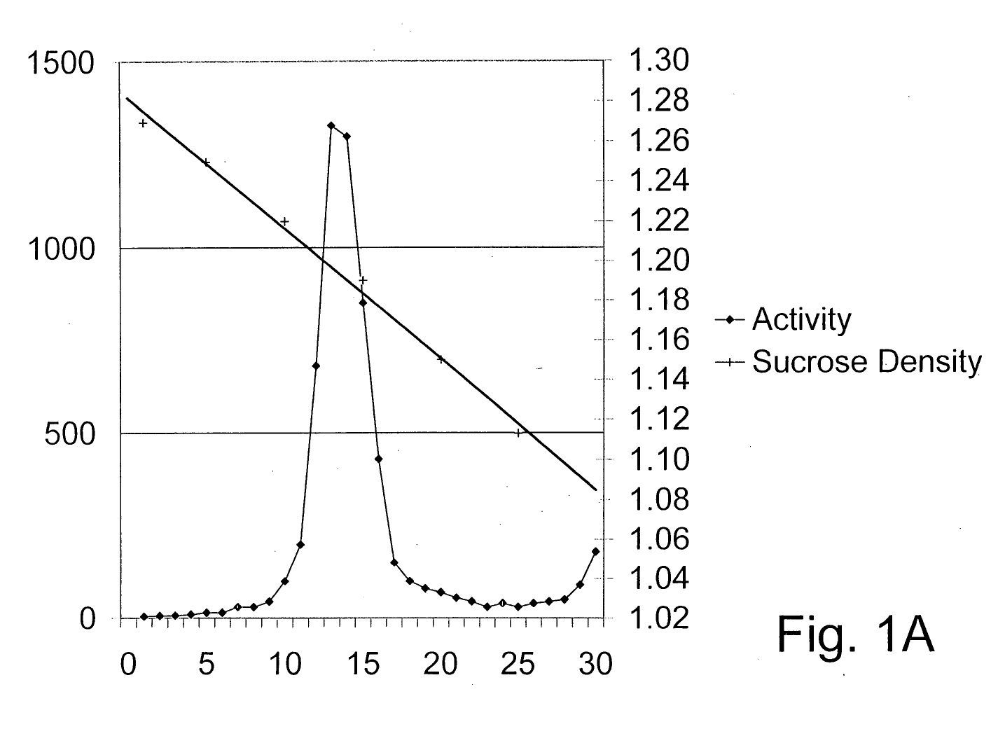

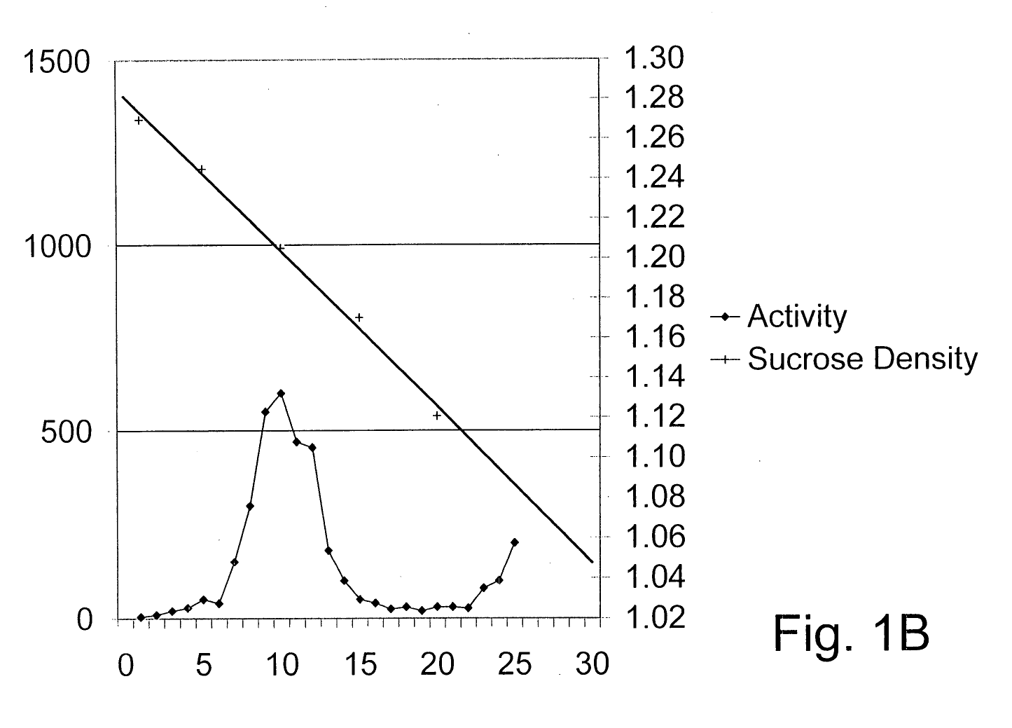

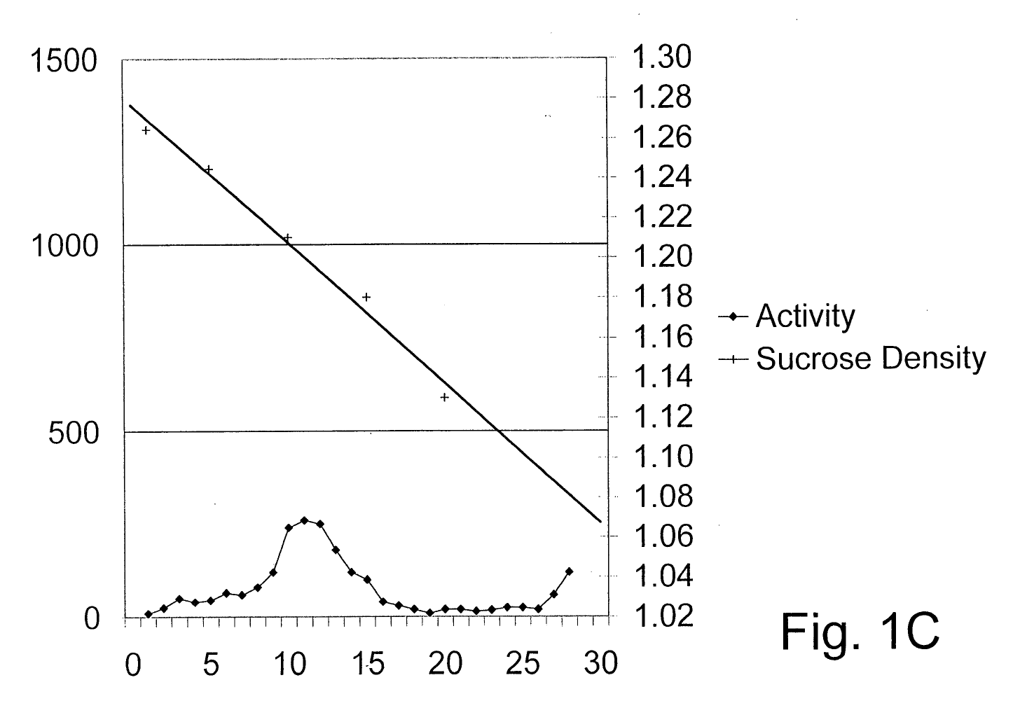

As shown in FIGS. 1A-1C, superoxide dismutase (SOD) from a vegetal source has a significant decreasing effect on mycoplasma growth. SOD was included in the medium at the first of the five day experiment. The SOD was purified from melon plant material. The control shown in FIG. 1A had a peak of about 1200 counts. The experimental treated with 50 μg/ml SOD had a peak of around 500 counts, while the experimental treated with 100 μg/ml SOD had a peak of around 250 counts.

These results are assumed to represent the ability of the SOD to reduce oxidative stress by converting superoxide radicals into hydrogen peroxide, which is further converted into water and oxygen by serum and cell catalases.

### EXAMPLE 2

The above method was modified by infecting the cells with HIV 1—Lai two weeks prior to the experiment, and glutathione and ascorbic acid (2:1 weight ratio, Thyogen Corp Ultrathione™) substituted for SOD.

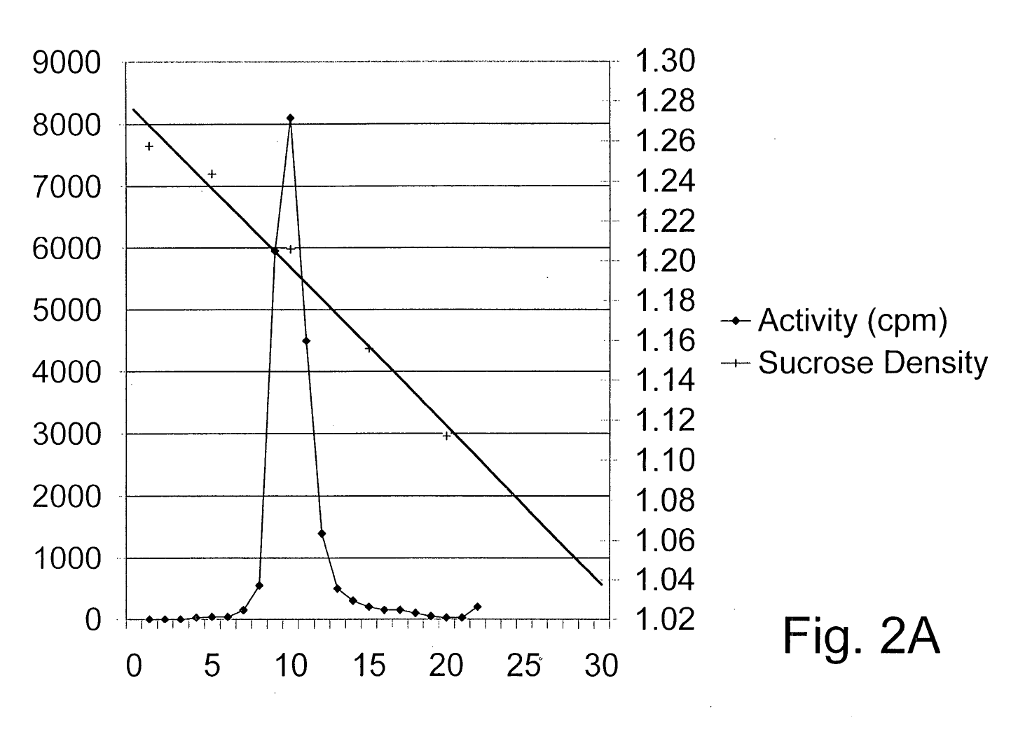

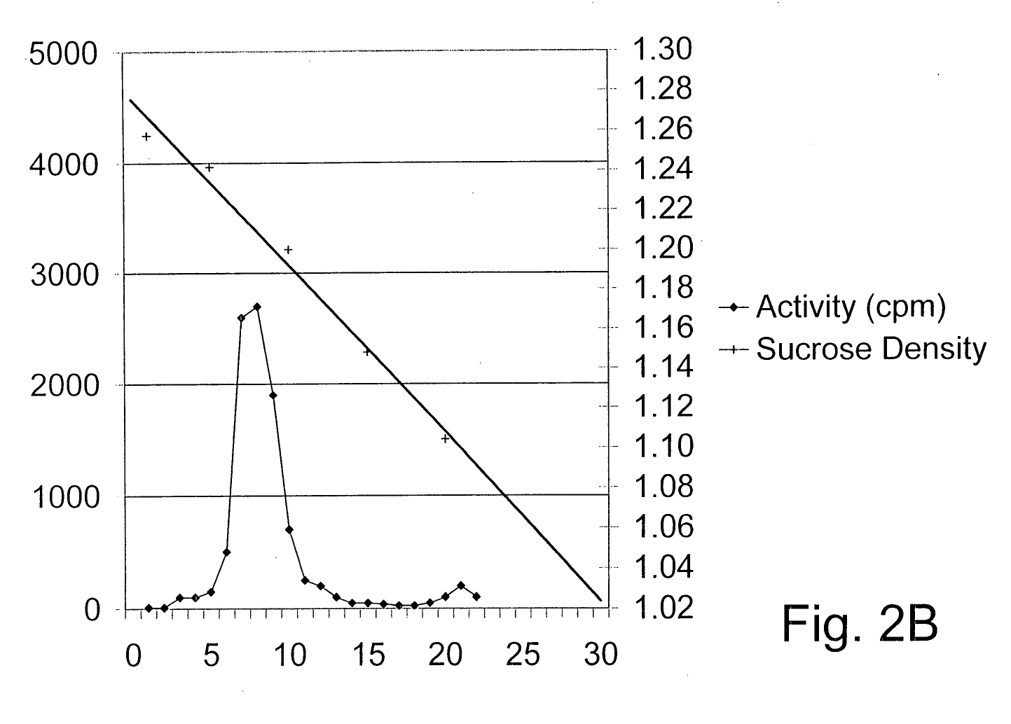

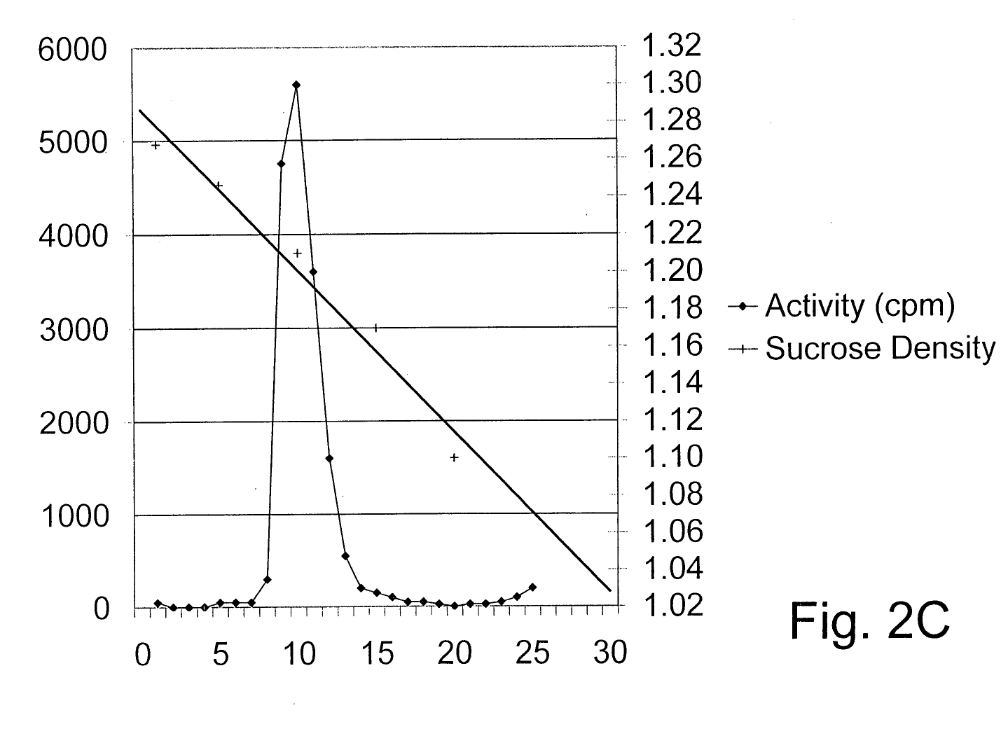

As shown in FIGS. 2A-2C, glutathione has a significant effect on mycoplasma growth which has an optimal suppressive dose of 1 mg/ml. The control shown in FIG. 2A had a peak of about 7000 counts. The experimental treated with 1 mg/ml glutathione had a peak of around 2700 counts, while the experimental treated with 3 mg/ml glutathione had a peak of around 5500 counts.

It is assumed that an HIV factor, possibly the Tat protein, induces mycoplasma activity, by raising the oxidative stress. The mycoplasma activity itself may also induce oxidative stress. The glutathione in an optimal concentration counteracts this oxidative stress, and therefore reduces mycoplasma growth as measured by 3H-uracil incorporation into DNA.

The higher dose of glutathione may have resulted in reduced inhibition of mycoplasma because this dose, 3 mg/ml, is close to the dose toxic to the host cells.

It should be understood that the preferred embodiments and examples described herein are for illustrative purposes only and are not to be construed as limiting the scope of the present invention, which is properly delineated only in the appended claims.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. A method of treating a chronic animal or human pathology associated with infection by a prokaryotic microorganism lacking a rigid cell wall, comprising
administering a pharmacologically active composition to efficaciously target said prokaryotic microorganism infecting the animal or human, said composition comprising at least one antibiotic and at least one antioxidant, the at least one antioxidant being present in an efficacious amount to reduce prokaryotic microorganism lacking a rigid cell wall infection when coadministered with said at least one antibiotic, said at least one antioxidant altering an environment within the animal or human with respect to oxidative status within a range which reduces activity of said prokaryotic microorganism, independent of said antibiotic.

28. The method according to claim 27, wherein said pharmacological composition comprises an antibiotic drug, and said altering step is performed by administering at least one antioxidant, the at least one antioxidant is present in an efficacious amount to reduce activity of said prokaryotic microorganism.

29. The method according to claim 27, wherein said administering step comprises administering at least two antibiotic drugs each targeting a separate metabolic pathway of said prokaryotic microorganism.

30. The method according to claim 27, further comprising the step of detecting a presence of said prokaryotic microorganism.

31. The method according to claim 27, further comprising adjusting said administering step based on a determination of metabolic activity of said prokaryotic microorganism.

32. The method according to claim 27, further comprising the step of adjusting said altering step based on a determination of metabolic activity of said prokaryotic microorganism.

33. The method according to claim 27, further comprising the step of adjusting said administering step and said altering step based on a determination of metabolic activity of said prokaryotic microorganism.

34. The method according to claim 27, wherein said pharmacological agent is selected from the group consisting of one or more of a tetracycline derivative and a fluoroquinolone antibiotic.

35. The method according to claim 27, wherein said antioxidant is selected from the group consisting of one or more of glutathione and superoxide dismutase.

36. (canceled)

37. (canceled)

38. (canceled)

39. (canceled)

40. The method according to claim 27, wherein said antibiotic comprises one or more of minocycline and doxycycline.

41. The method according to claim 27, wherein said antibiotic comprises one or more of ciprofloxacin and sparfloxacin.

42. The methyod according to claim 27, wherein said antibiotic comprises doxycycline and ciprofloxacin and said antioxidant comprises glutathione and ascorbic acid.

43. The method according to claim 27, further comprising administering at least one antiretroviral active agent.

44. A method of treating a human having a chronic infection with a prokaryotic microorganism lacking a rigid cell wall, comprising administering a synergistic and efficacious combination of at least one antibiotic and at least one antioxidant, said at least one antioxidant altering an environment within the animal or human with respect to oxidative status within a range which reduces activity of said prokaryotic microorganism, independent of said antibiotic, and wherein said antioxidant and said antibiotic are each administered in an amount which is substantially non-toxic to the human.

45. The method according to claim 44, wherein the antibiotic is administered in an amount which would be ineffective in eliminating the prokaryotic microorganism infection alone.

46. The method according to claim 44, further comprising the step of simultaneously treating the human for a retroviral infection.
